(19) United States
(12) Patent Application Publication
(10) Pub. No.: US 2025/0161944 A1
(43) Pub. Date: May 22, 2025

(54) CARTRIDGE WITH LAB ON PAPER TYPE SENSORS AND A METHOD OF MEASUREMENT WITH THEIR USE

(71) Applicant: POLITECHNIKA WARSZAWSKA, Warszawa (PL)

(72) Inventors: Kamil ZUKOWSKI, Reszel (PL); Katarzyna TOKARSKA, Warszawa (PL); Ilona GRABOWSKA-JADACH, Jablonna (PL); Michal CHUDY, Zyrardów (PL); Artur DYBKO, Warszawa (PL)

(21) Appl. No.: 18/835,023

(22) PCT Filed: Oct. 16, 2023
(86) PCT No.: PCT/PL2023/050085

§ 371 Date: Aug. 1, 2024

(30) Foreign Application Priority Data
Nov. 23, 2022 (PL) P.442916

(51) Int. Cl. B01L 3/00; G01N 30/92; G01N 33/22

(52) U.S. Cl. CPC B01L 3/5055; G01N 30/92; G01N 33/227; B01L 2200/025; B01L 2200/10; B01L 2300/043; B01L 2300/044; B01L 2300/047; B01L 2300/0672; B01L 2300/069; B01L 2300/0816; B01L 2300/126; B01L 2400/0406

(57) ABSTRACT

The invention relates to a cartridge with lab on paper type sensors, characterized in that it consists of an upper cartridge housing (6) and a lower cartridge housing (10), detachably connected by a hinge (8), and a chromatography paper with the printed architecture of lab on paper sensors type (9) placed between the housings (6, 10), wherein the upper cartridge housing (6) is divided into an outer zone on which a slot (3) is located to which a blister with a solvent (1) is attached by a double-sided adhesive tape (2), and an inner zone containing a centrally positioned slot (7) equipped with a porous foil (5) around which symmetrically arranged openings (4) are located, additional aligning elements are located on the corners of each of the elements (6, 9, 10), and on the lower cartridge housing (10), at the inner side thereof, a spacer structure (11) is located of a shape being an envelope of the area that includes the central slot (7) and openings (4) so that when assembled, the cartridge forms a measuring system. The invention also relates to a measurement method carried out using said cartridge and its use for detection of explosives.

The invention relates to a cartridge with lab on paper type sensors and a measurement method with their use.

The cartridge with lab on paper type sensors was designed to test or analyze materials by determining their chemical or physical properties.

The dynamic development of science is perceptible in the field leading to full integration of all steps related to collecting the sample, its preparation for analysis, conducting of the measurement and further sending and presenting a result.

In the scientific literature, such type of device is called laboratory on a chip ang. lab-on-a-chip. The main advantage of the devices being constructed is a significant reduction in the sample volume of the analyzed sample, which is expressed in micro or nanoliters, as well as a reduction in the volume of reagents required during the measurements. This contributes e.g. to cutting down the time and reducing the cost of performing a single analysis [1].

In addition to the above technique, a so-called lab on paper is a new field. In the lab on paper technique, it is assumed that a flow microsystem architecture is manufactured on a hydrophilic substrate (e.g. blotting paper for filtration) by introducing hydrophobic barriers. The tested sample is transported by employing capillary forces of the substrate. In specific areas of the lab on paper, chemical indicators are placed, e.g. to show color changes depending on the measured analyte. In the cartridge, a blister containing a solution for dissolving the tested sample is mounted. The blister is pierced with needles formed in the cartridge base [2],

Patent description KR102447967B1 discloses a solution for a laboratory system with a lab on paper sensor containing a heating system intended for the isothermal amplification method, and a method for detecting multiple nucleic acids.

Patent description U.S. Pat. No. 11,385,183B2 discloses a method for detecting heavy metals using a bio-active sensor on paper that is inkjet printed with sol-trapped reagents to allow colorimetric test of enzyme activity. The test on the bioactive paper is capable of detecting a range of heavy metals, both individually and in mixtures, in just 10 minutes. An advantage of the test is no interference from metal ions such as Na⁺ or K⁺, and also the capability to be used for the detection of heavy metals in potable or lake water samples and delivering quantitative data, that were consistent with values obtained by atomic absorption.

Foregoing works describe the use and technologies for the preparation of sensors using chromatography paper, and also indicate the possibility of using lab on paper sensors for the detection of analytes in a liquid form. Therefore, the tested sample must be initially prepared (dissolved), and then applied to the sensor, or the sensor must be immersed in the sample.

The technical problem solved by the present invention is to obtain a solution that provides a completely integrated measurement process employing chemical sensors. The need to integrate in one measuring system a solution needed to prepare a liquid sample, a sensor, and a cartridge was noted. The solvent solution placed in the blister causes dissolution of the tested sample, and then it diffuses through the spacer enabling its contact with the chromatography paper, on which the chemical sensors in the form of the lab on paper have been produced.

The invention relates to a cartridge with lab on paper type sensors, characterized in that it consists of an upper cartridge housing and a lower cartridge housing, detachably connected by a hinge, which is situated on the edge of the cartridge housing, and a chromatography paper with the printed sensors architecture of lab on paper type placed between the housings,
wherein the upper cartridge housing is divided into an outer zone, on which a slot is located, to which a blister with a solvent is attached by a double-sided adhesive tape, and an inner zone containing a centrally positioned slot equipped with a porous foil, around which symmetrically arranged openings are located,
wherein the shape of the inner zone of the upper cartridge housing corresponds to the shape of the chromatography paper and lower cartridge housing,
where additional aligning elements are located on the corners of each of the elements, and on the lower cartridge housing, at the inner side thereof, a spacer structure is located of a shape being an envelope of the area that includes the central slot and openings, so that when assembled, the cartridge forms a measuring system.

In one embodiment of the solution according to the invention, the cartridge is characterized in that the inner zone of the upper cartridge housing has four openings arranged symmetrically in relation to the central slot, so that they form a clover-leaf shape, which corresponds to the spacer structure located on the lower cartridge housing. The cartridge according to the invention has tapered needles on the inner edges of the slot, which, when the cartridge is assembled, pierce the blister with a solvent.

In the cartridge according to the invention, the chromatography paper with the printed sensors architecture of the lab on paper type is additionally immobilized by four cartridge alignment elements, which at the same time comprise latches compatible with their counterparts executed in the lower cartridge housing.

Preferably, the cartridge is manufactured by a micro-milling method in polyvinyl chloride.

Another subject-matter of the invention is the method of measurement carried out by using the cartridge according to the invention, the method being characterized in that the sample, which is constituted by a solid material, is placed in the slot, then the symmetrical position of the upper housing, lower housing and blotting paper is set by means of alignment openings, then the cartridge is closed, and during closing of the cartridge, the conical needles pierce the blister with a solvent, the sample dissolves and the obtained solution is transported by the generated capillary forces to the sensors placed on the paper, that change color under the influence of the sample, wherein the reading of the result is realized through the openings.

Preferably, the solvent placed in the blister is a mixture of acetone and distilled water in a proportion 50:50%.

The cartridge according to the invention is applicable to the detection of explosives. The advantage of the solution according to the invention consists in obtaining a fully integrated measurement process using the chemical sensors. This solution integrates in one instrument a solvent solution needed to prepare a liquid sample, a sensor with an indicator immobilized on chromatography paper with the printed sensors architecture lab on paper type, into which the dissolved sample diffuses, and a location where the result is read in the form of color change or fluorescence of the sample.

The advantage of the solution according to the invention consists in obtaining a completely integrated measurement process using the chemical sensors.

The subject-matter of the invention is illustrated in the drawings, wherein:

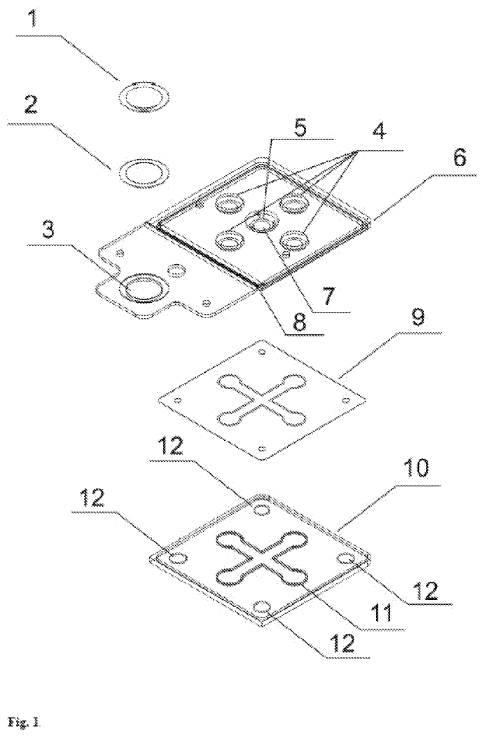

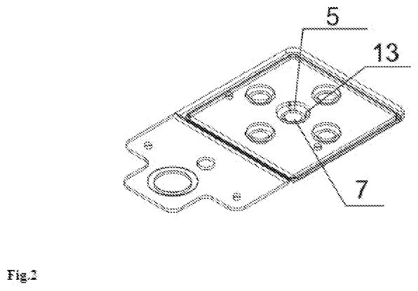

FIGS. 1-2 shows a schematic view of the structure of the cartridge according to the invention.

The subject-matter of the invention is presented in more detail in the preferred embodiments, which do not limit its scope.

### LIST OF REFERENCE NUMBERS

1—blister with a solvent
2—adhesive tape
3—slot
4—opening
5—porous foil
6—upper cartridge housing
7—slot
8—hinge
9—lab on paper sensor
10—lower cartridge housing
11—spacer structure
12—alignment elements
13—tapered needles

### Example 1. Cartridge Design and Sample Testing Method

The cartridge with lab on paper type sensors consists of a blister with solvent 1, which is placed in the slot 3 by means of a double-sided adhesive tape 2, a lower cartridge housing 10 made of polyvinyl chloride, an upper cartridge housing 6 made of polyvinyl chloride, chromatography paper with the printed sensors architecture of lab on paper type 9, a hinge 8, openings 4 for reading the result of color changes of the used lab on paper sensors 9, a porous foil 5, where the dissolution of the tested sample takes place, a slot 7 for placing the tested sample and a spacer structure 11 that distances the chromatography paper from the surface of the lower cartridge housing 10. In addition, on the inner edges thereof, the slot 3 has conical needles 13, which serve for piercing the blister 1 upon closing the cartridge.

The tested sample is placed in the slot 7, the cartridge is closed thanks to the flexible hinge 8, the blister 1 is punctured, the solvent solution from the blister cause dissolution of the sample and through the porous foil 5 to the chromatography paper with lab on paper sensors 9 is delivered. The solution prepared in this manner, due to capillary forces reaches the locations where chemical indicators are placed which exhibit changes in their optical properties depending on the concentration of the analytes tested (this can be a color change or fluorescence). The state of the lab on paper sensors is observed through openings 4.

### Example 2. Process of Making a Cartridge

The cartridge housing designed for mounting lab on paper sensors consists of two parts—the upper part 6 and the lower part 10, both made by micro-milling in PVC using the Datron Neo+ Series 2 milling device. All milling operations were performed at a spindle speed of 19,000 rpm. In order to minimize the risk of excessive heating of the tool, the milling was carried out in an ethanol mist cooling shield. The upper cartridge housing 6 has a maximum thickness of 2.8 mm and consists of two zones.

The structures in the upper cartridge housing 6 were made on both its sides by milling. First, the structures on the inside of the upper cartridge housing 6 were made. In the main zone of a square shape (38×38 mm), there are four openings 4 with a diameter of 5 mm, intended to enable reading an analysis result, and a central slot 7 for introducing a solid sample and its dissolving. The structures located on the inside of the upper cartridge housing 6 fix and press the chromatography paper with the printed sensors architecture of the lab on paper type 9. The chromatography paper with printed sensors architecture of the lab on paper type 9 is additionally immobilized by means of the four alignment elements 12 that align the lab on paper cartridge, and which also function as latches compatible with their counterparts formed in the lower cartridge housing 10.

On the outer side of the upper cartridge housing 6, the slot 7 equipped with four conical-shaped needles which enable puncturing the blister was made. They were made with a milling cutter of a 0.5 mm diameter at the following parameters: a feed rate in the XY axes of 1500 mm/min, a plunge rate of 300 mm/min, a plunge step of 0.05 mm, and an overlap of 0.5. On the outer surface of the upper cartridge housing 6, two openings are also located, the position of which corresponds to the situation of the two stubs in the second zone of the upper cartridge housing. The second zone also includes the slot 3 for the blister with solvent 1 which is fixed there by means of the double-sided adhesive tape 2 securing the blister to the housing. Both zones are separated by the hinge 8, bending of which and then snapping the stubs into the openings causes the assembly of the upper cartridge housing 6 and its final functionality.

The surface in the main zone of the upper cartridge housing 6 was initially machined with a double-flute end milling cutter of 2 mm diameter. The milling device was set to an XY feed rate of 2000 mm/min, a plunge rate of 250 mm/min, a plunge step of 0.5 mm, and a tool diameter overlap of 50%. The final machining was performed with a double-flute end milling cutter of 1 mm diameter. The device was set to an XY feed rate of 2000 mm/min, a plunge rate of 250 mm/min, a plunge step of 0.2 mm, and a tool diameter overlap of 75%. The processing of the surface in which the slot 3 of the blister with solvent 1 was placed was performed with a double-flute end milling cutter of 2 mm diameter. The device was set to an XY feed rate of 2000 mm/min, a plunge rate of 250 mm/min, a plunge step of 0.5 mm, and a tool diameter overlap of 50%. In the last stage, the plunge step was reduced to 0.2 mm and the tool diameter overlap was increased to 75% to obtain the highest quality surface at the optimal time of the entire process. The openings in the upper cartridge housing 6 were made in several steps with a double-flute end milling cutter of 1 mm diameter and a plunge step of 0.2 mm.

The lower cartridge housing 11 sized 38×38 mm and with the 2.8 mm height at the highest point was also made by milling in PVC. In its central part, a trefoil-shaped structure is located that distances the chromatography paper with the printed sensors architecture of the lab on paper type 9 from the lower surface of the lower cartridge housing 11. A double-flute end milling cutter of 1 mm diameter was used to make the inner structure. The device was set to an XY feed rate of 2000 mm/min, a plunge rate of 250 mm/min, a plunge step of 0.35 mm, and a tool diameter overlap of 65%.

After milling all the elements, the plates were washed in a detergent and thoroughly rinsed with deionized water.

### Example 3. Method of Detecting Explosives

The cartridge with lab on paper sensors was used to detect explosives, e.g. trotyl. For this purpose, a sample in solid form was placed in the slot 7 in the central point of the cartridge. The blister with solvent 1 contained a mixture of acetone and distilled water in proportion 50:50%. After the cartridge was closed by means of the flexible hinge 8, the blister with a solvent 1 was punctured, as a result of which the solvent reached the tested sample of the explosive. The solution resulting from dissolving the explosive is transported by capillary forces to the indicator immobilized on chromatography paper with the printed sensors architecture of lab on paper type 9. An example of such an indicator may be 3-aminopropyltriethoxysilane (APTES). APTES is a compound containing functional primary amino groups. As a result of the Meisenheimer reaction, trotyl forms a colored complex with the amino groups of APTES, therefore the lab on paper sensor changes its natural color to red, which indicates the detection of an explosive.

### LITERATURE

1. Jaime Castillo-León, Winnie E. Svendsen, Lab-on-a-Chip Devices and Micro-Total Analysis Systems, A Practical Guide, Springer 2015
2. Jeong Hoon Lee ed., Paper-Based Medical Diagnostic Devices: As a Part of 10 Bioanalysis-Advanced Materials, Methods, and Devices, Springer 2021

1. A cartridge with lab on paper type sensors, characterized in that it consists of an upper cartridge housing (6) and a lower cartridge housing (10), detachably connected by a hinge (8), which is situated on the edge of the cartridge housing (6), and a chromatography paper with the printed sensors architecture of lab on paper type (9) placed between the housings (6,10),
wherein the upper cartridge housing (6) is divided into an outer zone, on which a slot (3) is located, to which a blister with a solvent (1) is attached by a double-sided adhesive tape (2), and an inner zone containing a centrally positioned slot (7) equipped with a porous foil (5) around which symmetrically arranged openings (4) are located,
wherein the shape of the inner zone of the upper cartridge housing (6) corresponds to the shape of the chromatography paper (9) and the lower cartridge housing (10),
where additional aligning elements are located on the corners of each of the elements (6, 9, 10), and on the lower cartridge housing (10), at the inner side thereof, a spacer structure (11) is located of a shape being an envelope of the area that includes the central slot (7) and openings (4)
so that when assembled, the cartridge forms a measuring system.

2. The cartridge according to claim 1, characterized in that the inner zone of the upper cartridge housing (6) has four openings (4) arranged symmetrically in relation to the central slot (7), so that they form a clover-leaf shape, which corresponds to the spacer structure (11) located on the lower cartridge housing (10).

3. The cartridge according to any one of claims 1-2, characterized in that has the tapered needles (13) on the inner edges of the slot (3) have conical needles (13) which, when the cartridge is assembled, pierce the blister with a solvent (1).

4. The cartridge according to any one of claims 1-3, characterized in that the chromatography paper with printed sensor architecture of the lab on paper type (9) is additionally immobilized by four alignment elements (12) of the cartridge (6), which at the same time constitute latches compatible with their counterparts executed in the lower cartridge housing (10).

5. A method of measurement carried out by means of a cartridge according to claim 1, characterized in that the sample, which is constituted by a solid material, is placed in the slot (7), then the symmetrical position of the upper housing (6), lower housing (10) and chromatography paper (9) is set by means of alignment openings, then the cartridge is closed, and during closing of the cartridge, the conical needles (13) pierce the blister with a solvent (1), the sample is dissolved and the obtained solution is transported by the generated capillary forces to the sensors placed on the chromatography paper (9), that change color under the influence of the sample, wherein the reading of the result is realized through the openings (4).

6. Use of a cartridge according to claim 1 for detecting explosives.
